(19) United States
(12) Patent Application Publication
(10) Pub. No.: US 2017/0112361 A1
(43) Pub. Date: Apr. 27, 2017

(54) ENDOSCOPE CAP WITH DEFLECTING CHANNELS FOR ENDOSCOPIC THERAPY

(71) Applicant: COOK MEDICAL TECHNOLOGIES LLC, Bloomington, IN (US)

(72) Inventors: Vihar C. Surti, Winston-Salem, NC (US); John C. Sigmon, JR., Winston-Salem, NC (US)

(21) Appl. No.: 15/297,656

(22) Filed: Oct. 19, 2016

Related U.S. Application Data
(60) Provisional application No. 62/245,528, filed on Oct. 23, 2015.

(51) Int. Cl. A61B 1/00; A61B 1/018; A61B 17/295; A61B 17/3205

(52) U.S. Cl. CPC A61B 1/00098; A61B 17/3205; A61B 1/018; A61B 17/295; A61B 1/00137; A61B 2017/00269

(57) ABSTRACT

A medical device configured to facilitate a minimally invasive surgical procedure is provided. The medical device includes a housing having an attachment portion configured to removably engage with a scope. The medical device also includes a first deflector engaged with the housing and configured to engage with a distal end of a first treatment tool. The first deflector permits movement of the distal end of the first treatment tool independent of the housing. The medical device further includes a second deflector engaged with the housing and configured to engage with a distal end of a second treatment tool. The second deflector permits movement of the distal end of the second treatment tool independent of the housing.

### CROSS-REFERENCE TO RELATED APPLICATIONS

The present patent document claims the benefit of the filing date under 35 U.S.C. §119(e) of Provisional U.S. Patent Application Ser. No. 62/245,528 filed Oct. 23, 2015, which is hereby incorporated by reference.

### FIELD

The present disclosure relates to medical devices and more specifically to endoscopic submucosal dissection devices.

### BACKGROUND

The statements in this section merely provide background information related to the present disclosure and may not constitute prior art.

Endoscopic Submucosal Dissection (ESD) is a minimally invasive method for removing cancerous tissue in the gastrointestinal (GI) tract. ESD can be used when the tissue is cancerous within the first two layers of the GI wall: the mucosal and submucosal layers. If the cancerous tissue extends to any of the deeper layers of the GI wall, surgical or endoscopic full thickness resection may be required.

During a typical ESD procedure, an endoscope is inserted into a patient's mouth and through the GI tract until the distal end of the endoscope is adjacent to the cancerous tissue to be dissected. Treatment tools are then inserted through one or more working channels of the endoscope until their distal ends are also adjacent to the cancerous tissue. These treatment tools most commonly include forceps that are capable of grabbing and moving tissue and a cutting device that is capable of cutting into the tissue. The treatment tools can be manipulated by the physician along with the endoscope to grab the cancerous tissue and excise it from the surrounding healthy tissue of the GI wall.

Generally, the physician first uses a camera at the end of the endoscope to locate the cancerous tissue and then injects a saline solution or other injection fluid into the submucosal layer below the cancerous tissue. The fluid may be injected with a needle or other device that can be passed through or outside of the endoscope. The fluid creates a bubble beneath the cancerous tissue that causes the cancerous tissue to rise and protrude into the GI tract, thus making it easier for the physician to visualize and operate on/excise the cancerous tissue. Next, the physician inserts the cutting device into the working channel of the endoscope and an initial cut is made with the cutting device around the cancerous tissue. The cutting device is then moved left and right to make a series of small cuts around the entire cancerous area. Additional saline may be injected under the cancerous tissue during this process as necessary. This process is continued until the cancerous tissue has been completely excised from the surrounding, healthy tissue. Some endoscopes exist that include two working channels, thus allowing two treatment tools to be used simultaneously. Therefore, forceps can be used with the cutting device to grasp and lift the mucosal flap that was created by the abovementioned initial cut made by the cutting device. This step may increase the visibility of the cancerous tissue that needs to be excised, thus decreasing the difficulty of the procedure. However, endoscopes with two working channels are rarely used in practice for ESD procedures, and the end-terminal region flexibility of one- or two-channel scopes is limited in a manner that concomitantly limits the orientation and manipulability of tools (e.g., forceps, cutting tool).

There are various other challenges for current ESD procedures. The procedure is not simple, and often takes even experienced physicians over 2 hours to complete. Due to the high degree of difficulty, complications with this procedure such as bleeding and perforation are a risk. This procedure is difficult in part due to the anatomical limitations of the GI tract. Because this procedure is minimally invasive and is not done with open surgery, the physician has limited working space within a small, winding GI tract to maneuver the endoscope, cutting device, and sometimes forceps. Additionally, the physician must manipulate the distal end of the treatment tools from a point external the patient while the cancerous tissue may be several hundred centimeters along the GI tract. Due to the length of the treatment tools and/or the limitations of the endoscope manipulability, the physician may encounter difficulty controlling the distal ends of the treatment tools and making the necessary precise movements to excise the cancerous tissue without inadvertently cutting a vessel or other part of the GI wall. Also, the physician relies on a camera located at the distal end of the endoscope to perform the procedure. Because the cutting device (and, sometimes, forceps) extend through working channels of the endoscope, the distal ends of the treatment tools tend to at least partially obstruct the visibility of the camera, thus making it more difficult for the physician to accurately excise only the cancerous tissue and not inadvertently cut a vessel or perforate through a deeper layer of the GI wall. Additionally, because the endoscope frequently moves (and often must be moved along) with the treatment tools as they are being manipulated, the physician may have difficulty positioning the endoscope so that the target tissue area remains visible while still having the treatment tools in a position to work on the target area. Therefore, the physician must reposition the endoscope and tools multiple times throughout the procedure, thus increasing the time to complete the procedure and the degree of difficulty. Additionally, because the distal ends of the treatment tools are restricted by the working channels of the endoscope, the distal ends of the treatment tools may have limited range of motion, thus limiting their effectiveness.

Thus, it is desirable to decrease the degree of difficulty of performing an ESD while increasing the effectiveness and range of motion of the treatment tools.

### SUMMARY

In one form of the present disclosure, a medical device configured to facilitate a minimally invasive surgical procedure is provided. The medical device comprises a housing comprising an attachment portion configured to removably engage with a scope. The medical device further comprises a first deflector engaged with the housing, the first deflector configured to engage with a distal end of a first treatment tool, the first deflector permitting movement of the distal end of the first treatment tool independent of the housing. The medical device further comprises a second deflector engaged with the housing, the second deflector configured to engage with a distal end of a second treatment tool, the second deflector permitting movement of the distal end of the second treatment tool independent of the housing.

In another form of the present disclosure, the medical device may further include a scope removably engaged with the attachment portion of the housing. The medical device may further include the housing, the first deflector, and the second deflector being configured such that actuating movement of the first deflector relative to the housing occurs along a first plane and actuating movement of the second deflector occurs along a second plane. The first plane may be substantially orthogonal to the second plane. The medical device may also include a first accessory catheter attached to the housing and a first working channel extending through the first accessory catheter, the housing, and the first deflector, wherein the first working channel is configured to removably receive and allow operation therethrough of a first treatment tool. The medical device may further include a second accessory catheter attached to the housing and a second working channel extending through the second accessory catheter, the housing, and the second deflector, wherein the second working channel is configured to removably receive and allow operation therethrough of a second treatment tool. Additionally, the medical device may comprise the first treatment tool comprising a cutting tool and the second treatment tool comprising a forceps, with each disposed and operably through one of the first deflector and the second deflector. The medical device may further comprise the first deflector comprising first and second pull members, the first pull member configured to rotate the first deflector in a first direction and the second pull member configured to rotate the first deflector in a second direction, wherein rotation of the first deflector is configured to cause corresponding rotation of the distal end of the first treatment tool, wherein the second direction is diametrically opposed to the first direction. The medical device may further comprise the second deflector comprising third and fourth pull members, the third pull member configured to rotate the second deflector in a third direction and the fourth pull member configured to rotate the second deflector in a fourth direction, wherein rotation of the second deflector is configured to cause corresponding rotation of the distal end of the second treatment tool, wherein the third direction is diametrically opposed to the fourth direction.

In yet another embodiment of the disclosure, a method of removing tissue from a body lumen is provided. The method includes providing a scope cap, the scope cap comprising a housing comprising an attachment portion, the attachment portion configured to removably engage with a scope, a first deflector engaged with the housing, the first deflector configured to receive and allow operation therethrough of a distal end of a cutting device, the first deflector permitting movement of the distal end of the cutting device independent of the housing, and a second deflector engaged with the housing, the second deflector configured to receive and allow operation therethrough of a distal end of a forceps, the second deflector permitting movement of the distal end of the forceps independent of the housing. The method also includes advancing the scope cap through the body lumen until the scope cap is adjacent to a target tissue to be removed from a surrounding tissue. The method further comprises inserting a cutting device through a first working channel that extends through and provides a path of mechanical communication through a first accessory catheter, the housing, and the first deflector, the first accessory catheter extending proximally from the housing and inserting the forceps through a second working channel that extends through and provides a path of mechanical communication through a second accessory catheter, the housing, and the second deflector, the second accessory catheter extending proximally from the housing. The method also comprises manipulating the second deflector to grab a portion of the target tissue with the forceps and separate the portion of the target tissue from the surrounding tissue and manipulating the first deflector to create a series of incisions in the target tissue. Additionally, the method comprises repeating the steps of manipulating the second deflector and manipulating the first deflector with independent and/or concurrent operation of the cutting device and/or forceps until the target tissue is excised from the surrounding tissue.

The method may further comprise providing a scope and attaching the scope cap to a distal end of the scope. The method may further include the first accessory channel extending proximally from the housing along and external to the scope and the second accessory channel extending proximally from the housing along and external to the scope.

In yet another aspect of the present disclosure, a system for performing a minimally invasive procedure is provided. The system comprises a cap, the cap including a housing comprising an attachment portion configured to removably engage with a scope. The cap further comprises a first deflector engaged with the housing, the first deflector configured to engage with a distal end of a first treatment tool and permitting movement of the distal end of the first treatment tool independent of the housing. The cap also comprises a second deflector engaged with the housing, the second deflector configured to engage with a distal end of a second treatment tool and permitting movement of the distal end of the second treatment tool independent of the housing.

The system may further comprise a scope, the cap removably attached to a distal end of the scope. The system may also include the cap further comprising a first accessory catheter attached to the housing and a first working channel extending and providing a path of mechanical communication through the first accessory catheter, the housing, and the first deflector, wherein the first working channel is configured to removably receive and allow operation therethrough of the first treatment tool. The system may also include the cap further comprising a second accessory catheter attached to the housing and a second working channel extending and providing a path of mechanical communication through the second accessory catheter, the housing, and the second deflector, wherein the second working channel is configured to removably receive and allow operation therethrough of the second treatment tool. Also, the system may comprise the housing, the first deflector, and the second deflector being configured such that actuating movement of the first deflector relative to the housing occurs along a first plane and actuating movement of the second deflector occurs along a second plane.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a drawing of an endoscope with an endoscope cap in accordance with the teachings of the present disclosure;

FIG. 2 is a detailed view of an endoscope cap;

FIG. 3A is a view of an endoscope cap with a cutting deflector in a neutral position;

FIG. 3B is a view of an endoscope cap with a cutting deflector at one end of its range of motion;

FIG. 3C is a view of an endoscope cap with a cutting deflector at another end of its range of motion;

FIG. 4 is a detailed view of an endoscope cap;

FIG. 5A is a view of an endoscope cap with a forceps deflector in a neutral position;

FIG. 5B is a view of an endoscope cap with a forceps deflector at one end of its range of motion; and

FIG. 6 is view of an alternative embodiment of the scope cap.

### DETAILED DESCRIPTION

The following description is merely exemplary in nature and is not intended to limit the present disclosure, application, or uses. It should be understood that throughout the drawings, corresponding reference numerals indicate like or corresponding parts and features. It should also be understood that various cross-hatching patterns used in the drawings are not intended to limit the specific materials that may be employed with the present disclosure. The cross-hatching patterns are merely exemplary of preferable materials or are used to distinguish between adjacent or mating components illustrated within the drawings for purposes of clarity.

As used herein, the phrase “configured to” (including “configured for”) is not aspirational, nor does it merely indicate a statement of intended use; rather, “configured to” describes specific structural limitations as expressly disclosed (including their legal equivalents) which will be understood by those of skill in the art as providing an effective structure and mechanism dictated by and particularly suited for the function described. In other words, the phrase “configured for” means that the structure configured for a given function and/or structural interface is effective for doing that as interpreted within the boundaries of the present specification and its legal equivalents. The terms “proximal” and “distal” are used herein in the common usage sense where they refer respectively to a handle/doctor-end of a device or related object and a tool/patient-end of a device or related object. The terms “scope” and “endoscope” refer to and include medical scopes that are configured to be directed into a patient body via a natural orifice or percutaneously (e.g., gastrointestinal endoscope, anuscope, laparoscope, etc.).

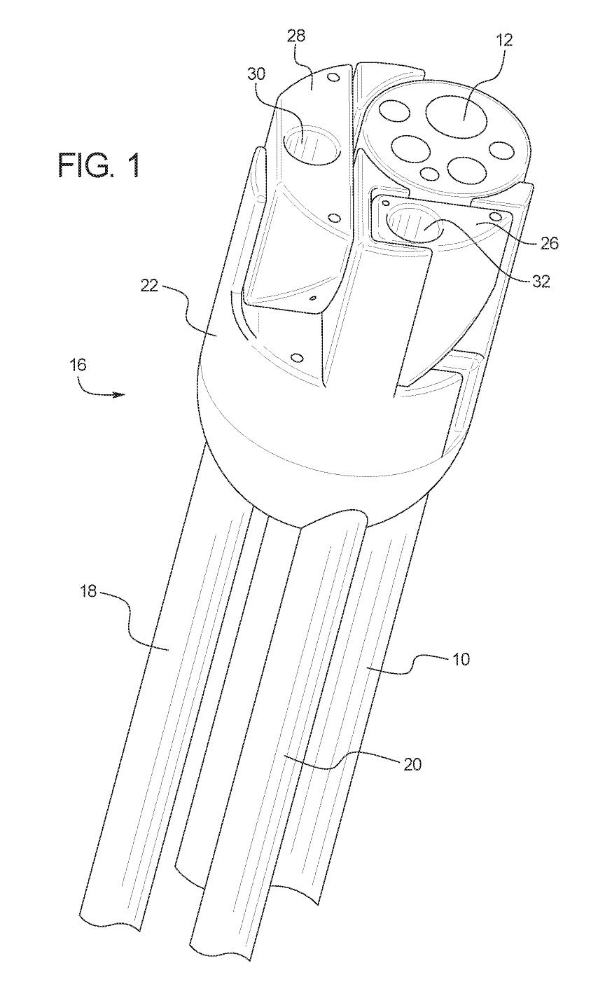

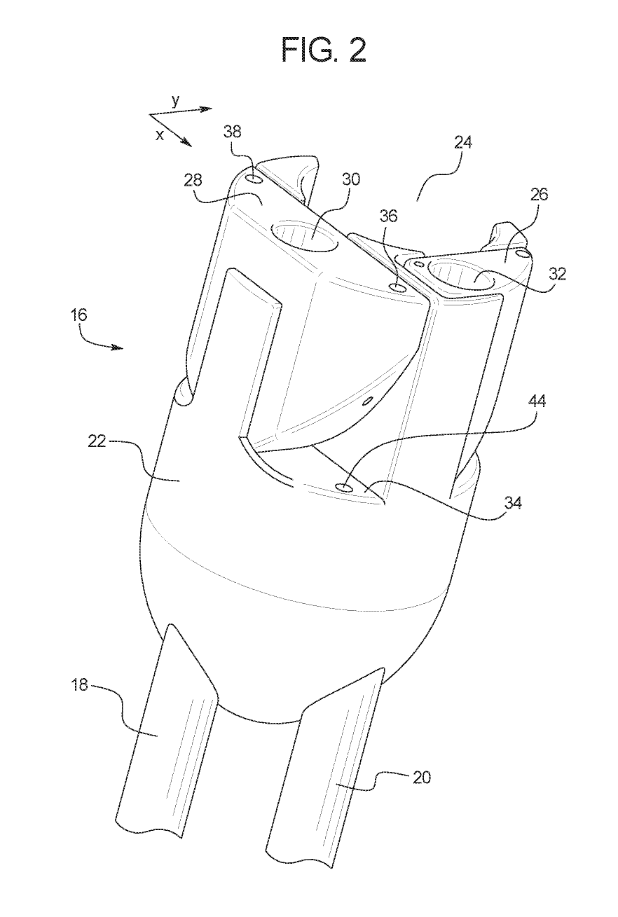

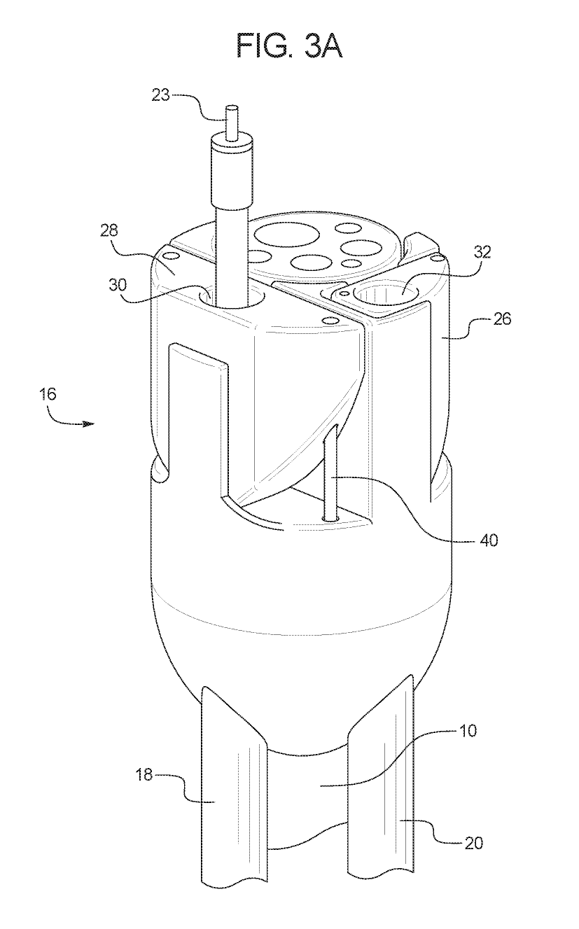

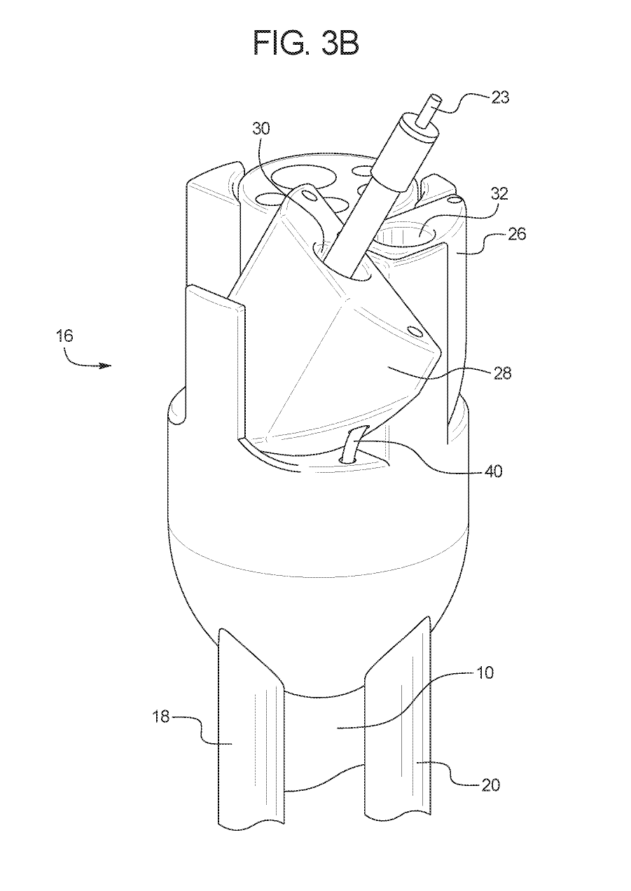

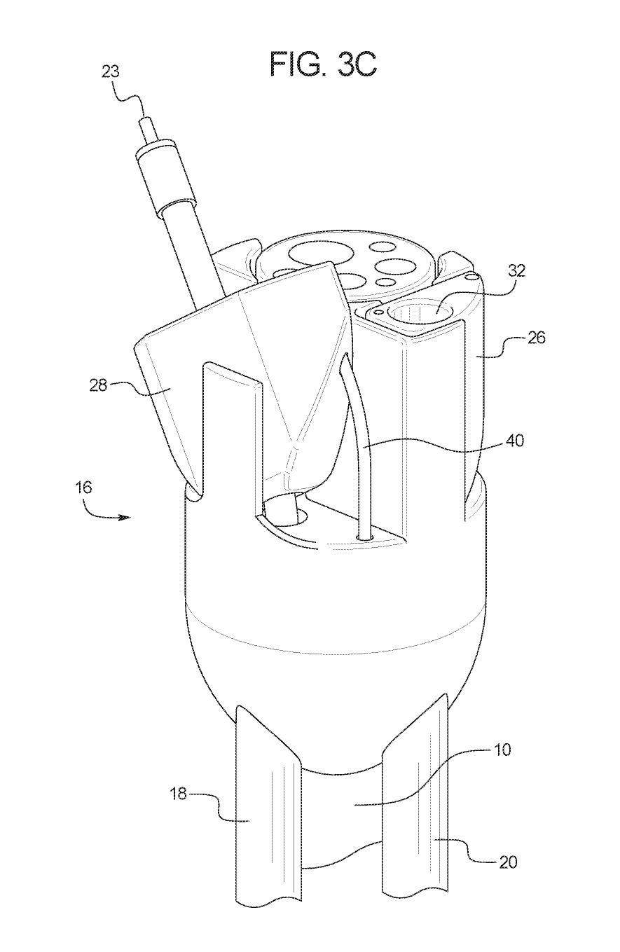

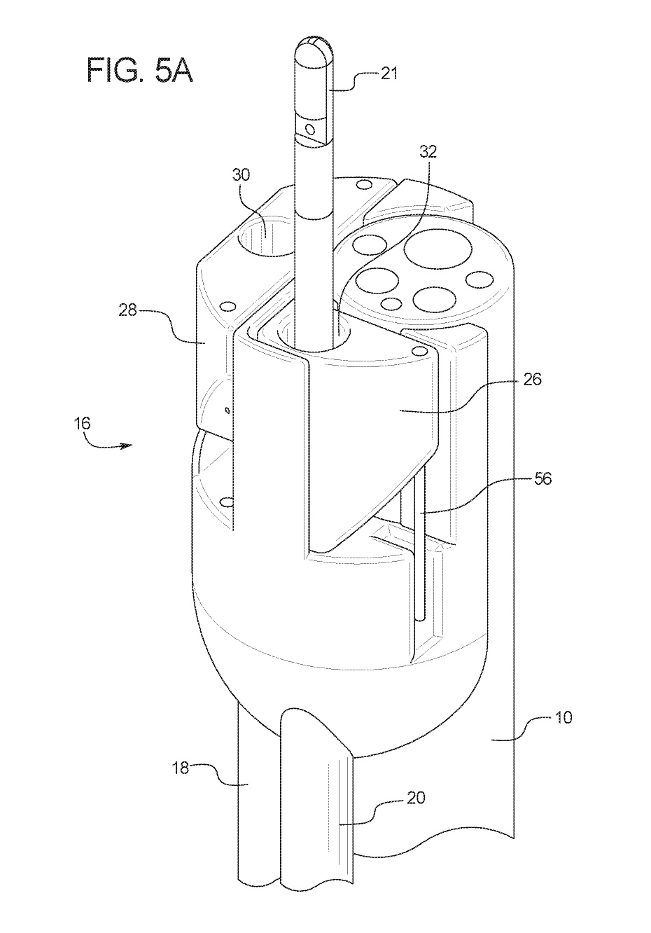

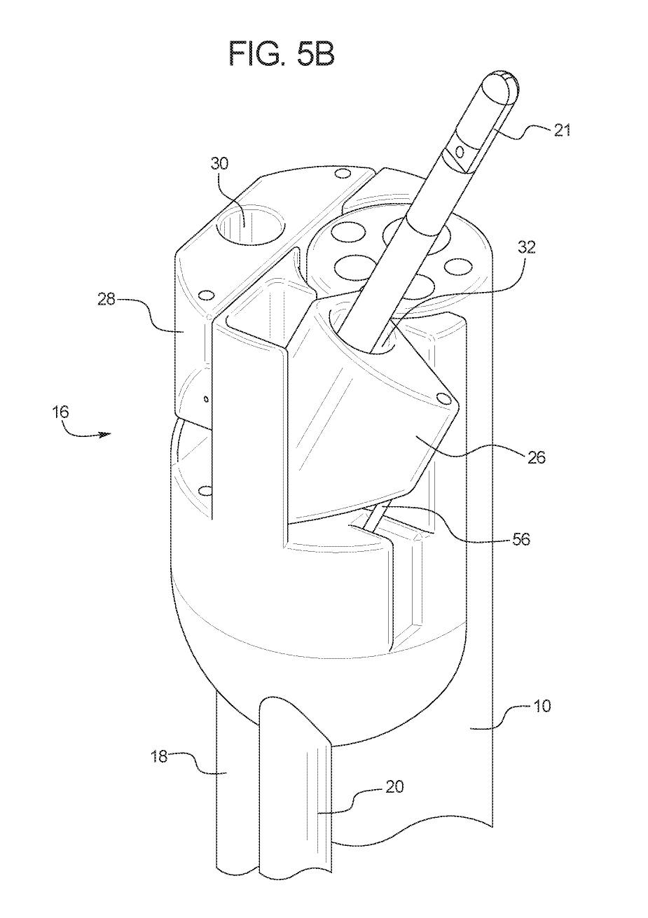

FIG. 1 shows a distal portion of an endoscope 10 with an endoscope channel 12. Attached to the endoscope 10 is an endoscope cap 16, which is shown in detail in FIG. 2. The cap 16 may include a housing 22 with an attachment portion 24 within which the endoscope 10 is designed to fit. In this embodiment, the attachment portion comprises a c-shaped opening within which the endoscope 10 may be friction force fit. This design allows the cap 16 to be easily attached and removed as desired. However, the cap 16 may be attached to the endoscope 10 in a variety of other methods well known in the art, including, but not limited to: a biocompatible adhesive, a strap wrapped around the cap 16 and the endoscope 10, mechanical fasteners, or clamps. The cap 16 may further include a first accessory catheter 18 and a second accessory catheter 20 attached to the housing 22. The accessory catheters 18, 20 ideally extend from the housing 22 along and external to the endoscope 10 to a point external the patient. The cap 16 may further include a forceps deflector 26 and a cutting deflector 28. A first working channel 30 may extend internally along the entire length of the first accessory catheter 18 through the housing 22 and the cutting deflector 28. A second working channel 32 may extend internally along the entire length of the second accessory catheter 20 through the housing 22 and the forceps deflector 26. Each working channel 30, 32 may receive a treatment tool. For example, the first working channel 30 may receive a forceps 21 (shown in FIGS. 5A and 5B) and the second working channel 32 may receive a cutting device 23 (shown in FIGS. 3A, 3B, and 3C), where the cutting device may be embodied as an electrocautery needle knife or other cutting device.

As shown in detail in FIG. 2, the cutting deflector 28 slides into a slot 34 in the housing 22 that engages the cutting deflector 28. The slot 34 ideally permits the cutting deflector 28 to move both forwards and backwards in the x-direction while preventing any movement in the y-direction. Ideally, the cutting deflector 28 is not directly secured to the housing 22 with the exception of some friction between the cutting deflector 28 and the slot 34 (and via one or more pull members described below), thus allowing the cutting deflector 28 to have free range of motion in the x-direction. However, the cutting deflector 28 may be attached to the housing 22 in other ways well known in the art. The cutting deflector 28 may include a first pull member channel 36 and a second pull member channel 38. A first pull member 40 (shown in FIGS. 3A, 3B, and 3C) may be disposed within the first pull member channel 36 and fixedly attached to the cutting deflector 28. A second pull member 42 (not shown), corresponding to the first pull member 40, may be disposed within the second pull member channel 38 and fixedly attached to the cutting deflector 28. The first pull member 40 may extend from the first pull member channel 36 through a first housing pull member channel 44 while the second pull member 42 may extend from the second pull member channel 38 through a second housing pull member channel 46 (not shown). Both pull members 40, 42 may then extend through the first accessory catheter 18 to a point external the patient. The pull members 40, 42 may extend through separate lumens within the first accessory catheter 18, through a single dedicated lumen for the pull members 40, 42, or even through the first working channel 30. Alternatively, the pull members 40, 42 may extend along and external to the first accessory catheter 18.

The pull members 40, 42 may be manipulated by the physician to move the cutting deflector 28 and, by extension, the distal end of the cutting device 23. FIGS. 3A, 3B, and 3C show the cutting deflector 28 in three different positions. FIG. 3A shows the cutting deflector 28 in its neutral position, while FIGS. 3B and 3C show the cutting deflector 28 at each end of the cutting deflector's 28 range of motion. To move the cutting deflector 28, and by extension the cutting device 23, from the position shown in FIG. 3A to the position shown in FIG. 3B, the physician may pull the first pull member 40 in a proximal direction. Because the distal end of the first pull member 40 is attached to the cutting deflector 28, the end of the cutting deflector 28 to which the first pull member 40 is attached will also be pulled in a proximal direction. This movement will cause the cutting deflector 28 to rotate about the axis of the first working channel 30 to the position shown in FIG. 3B. To move the cutting deflector 28 and cutting device 23 back towards original position shown in FIG. 3A or even further to the position shown in FIG. 3C, the physician may pull the second pull wire 42 in a proximal direction, thus causing the end of the cutting deflector 28 that is attached to the second pull member 42 to also move in a proximal direction. Thus, the cutting deflector 28 and cutting device 23 will once again rotate about the axis of the first working channel 30 back towards the position shown in FIG. 3A. Further movement of the second pull member 42 in the proximal direction will cause the cutting deflector 28 and cutting device 23 to rotate to the position shown in FIG. 3C. This movement can be repeated as necessary by the physician.

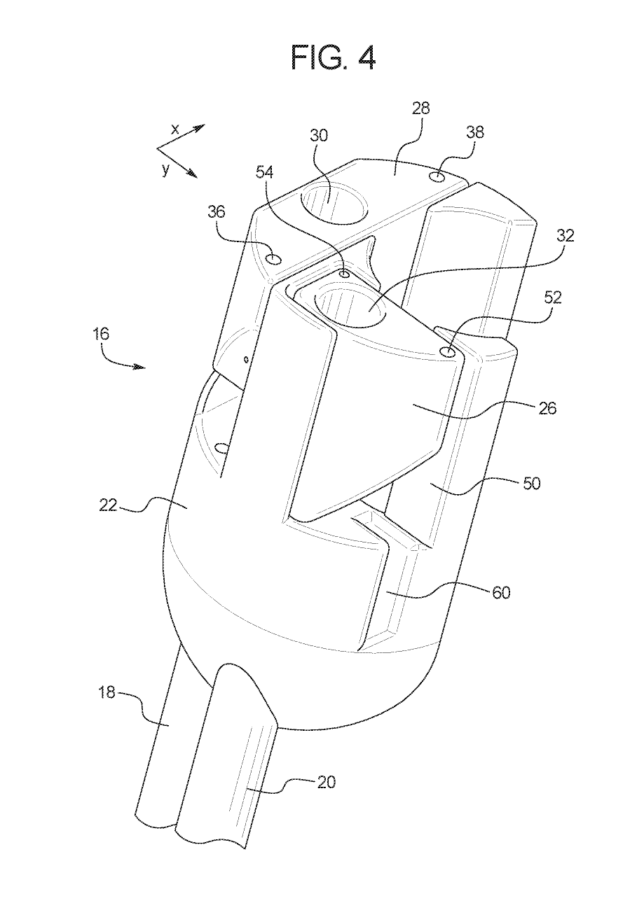

The forceps deflector 26 is constructed and operates in a manner similar to the cutting deflector 28. As shown in FIG. 4, the forceps deflector 26 slides into a slot 50 in the housing 22 that engages the forceps deflector 26. The slot 50 ideally permits the forceps deflector 26 to move in the y-direction while preventing any movement in the x-direction. Ideally, the forceps deflector 26 is not directly secured to the housing with the exception of some friction between the forceps deflector 26 and the slot 50, thus allowing the forceps deflector 26 to have free range of motion in the y-direction. However, the forceps deflector 26 may be connected to the housing 22 in other ways well known in the art. The forceps deflector 26 may include a third pull member channel 52 and a fourth pull member channel 54. A third pull member 56 (shown in FIGS. 5A and 5B) may be disposed within the third pull member channel 52 and attached to the forceps deflector 26 A fourth pull member 58 (not shown), corresponding to the third pull member 56, may be disposed within the fourth pull member channel 54 and fixedly attached to the forceps deflector 26. The third pull member 56 may extend from the third pull member channel 52 through third housing pull member channel 60 while the fourth pull member 58 may extend from the fourth pull member channel 54 through a fourth housing pull member channel 62 (not shown). Both pull members 56, 58 may then extend through the second accessory catheter 20 to a point external the patient. The pull members 56, 58 may extend through separate lumens within the second accessory catheter 20, through a single dedicated lumen for the pull members 56, 58, or even through the second working channel 32. Alternatively, the pull members 56, 58 may extend along and external to the second accessory catheter 20.

The pull members 56, 58 may be manipulated by the physician to move the forceps deflector 26 and, by extension, the distal end of the forceps 21. FIGS. 5A and 5B show the forceps deflector 26 at each end of the forceps deflector's 26 range of motion. The forceps deflector 26 is in its neutral position as shown in FIG. 5A. To move the forceps deflector 26, and by extension the forceps 21, from the neutral position in FIG. 5A to the position shown in FIG. 5B, the physician may pull the third pull member 56 in a proximal direction. Because the distal end of the third pull member 56 is attached to the forceps deflector 26, the end of the forceps deflector 26 to which the third pull member 56 is attached will also be pulled in a proximal direction. This movement will cause the forceps deflector 26 to rotate about the axis of the second working channel 32 to the position shown in FIG. 5B. To move the forceps deflector 26 and the forceps 21 back towards the original position shown in FIG. 5A, the physician may pull the fourth pull wire 58 in a proximal direction, thus causing the end of the forceps deflector 26 that is attached to the fourth pull wire 56 to also move in a proximal direction. Thus, the forceps deflector 26 and forceps 21 will once again rotate about the axis of the second working channel 32 back towards the position shown in FIG. 5A. This movement can be repeated as necessary by the physician.

For the purposes of simplified and clear illustrations, the pull members 40, 42, 56, 58 may not be shown in all drawing figures, but those skilled in the art will appreciate their location, structure, and function in view of the illustrations and narrative description that is provided.

As described above, the forceps deflector 26 may have a more limited range of motion than the cutting deflector 28. This may be an ideal design due to the required movements of the cutting device 23 and the forceps 21 during an ESD procedure. The forceps 21 are generally only required to grab and lift the mucosal flap or other tissue, thus only movement in a single direction from the neutral position of the forceps deflector 26 is required. However, the cutting device 23 is often used in a back and forth slicing motion, thus the ability of the cutting deflector 28 to move in both directions along the x-axis from its neutral position allows the cutting device 23 to reach and cut a larger portion of tissue, thus increasing the speed with which an ESD is performed. Alternatively, the cap 16 may be redesigned to give the forceps deflector 26 a greater range of motion than is shown in the current embodiment. Stated differently, in the embodiments illustrated, the range of motion of the forceps deflector 26 is along a maximal arc of about 180° with a single plane of reciprocation along an arc that—in function—is less than 180°; and, in the embodiments illustrated, the range of motion of the cutting deflector 23 is along a maximal arc of about 90° (between an axis parallel with and an axis orthogonal to the longitudinal axis of the device and of the endoscope), with a single plane of reciprocation along an arc that—in function—is less than 90°. The cutting deflector 23 and the forceps deflector 26 preferably are oriented so that their respective planes of arcing actuation are substantially or absolutely orthogonal to each other.

As mentioned above, in the illustrated embodiments, the cutting deflector 28 and forceps deflector 26 are not secured to the housing 22 with the exception of some frictional force between the housing 22 and the deflectors 26, 28. The deflectors 26, 28 are also maintained in position by their respective pull members 40, 42, 56, 58. Maintaining tension on all the pull wires 40, 42, 56, 58 throughout the procedure provides a continuing proximal force on the deflectors 26, 28 that assists in securing them in their slots 34, 50. Additionally, the forceps 21 and cutting device 23 extend through both the housing 12 and the deflectors 26, 28, thus providing additional support to maintain the housing 12 and deflectors 26, 28 together. These two factors, along with the frictional fit of the deflectors 26, 28 within the slots 34, 50, helps maintain the deflectors 26, 28 in their desired position.

The cap 16 has various advantages over the traditional endoscopes used in ESD procedures. First, because the cap 16 is attached to the external surface of the endoscope 10 and has separate working channels 30, 32, the working channel 12 of the endoscope 10 remain unused by the present device and useful for other tools and/or functions. These working channels may be used to inject fluids or use other working tools simultaneously with the cutting device 23 and forceps 21, thus eliminating the need to remove and insert several tools during the course of an ESD procedure. Additionally, the cap 16 at least partially removes the forceps 21 and cutting device 23 from the line of sight of the endoscope camera, thus increasing the effectiveness of the camera and the visibility of the target area. Further, the cap 16 can be used with standard endoscopes 10 that are common in the medical field. A physician merely has to press fit the cap 16 onto the distal end of a generic endoscope 10 and the device is ready to be used to perform an ESD. Additionally, the simplicity of the cap 16 design makes them inexpensive to manufacture and potentially disposable after a single use, providing advantages of time management, sterility, and cost-management for patients and care providers.

Perhaps most importantly, this design decreases the degree of difficulty of performing an ESD. Using a typical endoscope to perform an ESD means that the cutting device 23 and forceps 21 have limited range of motion at their distal ends. This factor often results in the physician having to move the endoscope and reposition the treatment tools repeatedly during the ESD. In the present embodiment, the deflectors 26, 28 provide an increased range of motion at the distal ends of the forceps 21 and cutting device 23. The increased range of motion allows the cutting device 23 and forceps 21 to excise a larger portion of the cancerous tissue while maintaining the endoscope in a fixed location. While the endoscope may still have to be moved and the treatment tools may have to be repositioned several times throughout the procedure, the amount of times that they need to be repositioned may be greatly reduced by the cap 16 in comparison to traditional endoscope designs. Additionally, this increased range of motion is possible without having to move the endoscope 10 along with the deflectors 26, 28. The endoscope 10, along with the endoscope's camera, may be statically positioned while the forceps 21 and cutting device 23 are manipulated. A relatively static endoscope camera makes it easier for the physician to see the cancerous or other target tissue, thus decreasing the difficulty of excising that tissue. Overall, the cap 16 provides the physician with the ability to grab and cut the target tissue simultaneously to keep the target area visible at all times and reachable in difficult anatomical positions, all while the endoscope 10 remains in a fixed location.

The pull members 40, 42, 56, 58 may be comprised of a variety of biocompatible materials. For example, the pull wires may be made of a metallic, biocompatible wire, a braided polymer suture, or ultra-high molecular weight braided polyethylene. The accessory catheters 18, 20 may also be comprised of a variety of biocompatible materials including, but not limited to, a coiled polymer tubing to allow for flexibility. The cap 16, including the deflectors 26, 28 are ideally made of a biocompatible polymer such as polycarbonate, nylon, PEEK, and thermoplastic resins, one of more or which may be constructed as being translucent or transparent in a manner that can be useful during visualization of an operating field. However, a variety of other well-known biocompatible material(s) may be used.

While using the above embodiment during an ESD procedure, the physician may begin by press fitting the cap 16 to the distal end of the endoscope 10. The endoscope 10, along with the cap 16 and the accompanying accessory catheters 18, 20 may then be inserted into a patient's mouth and advanced through the patient's GI tract. Once the distal end of the endoscope 10 has reached the target area where the cancerous tissue is located, the endoscope camera may be aimed at the cancerous tissue and the endoscope 10 may remain at that fixed location. Optionally, a fluid injection device may be advanced through the working channel 12 of the endoscope 10 until the fluid injection device extends past the distal end of the endoscope 10. The fluid injection device may be used to inject fluid in the submucosal layer of the GI wall underneath the cancerous tissue. This injection may raise the cancerous tissue from the surrounding GI wall, thus making it easier for the physician to excise the cancer. Next, the cutting device 23 and forceps 21 may both be inserted into and advanced along their respective working channels 30, 32. Alternatively, the cutting device 23 and forceps 21 may be preloaded in their respective working channels 30, 32 as the endoscope 10 and cap 16 are initially advanced along the patient's GI tract. Once the distal ends of the cutting device 23 and forceps 21 extend distally past the deflectors 26, 28, an initial cut into the cancerous tissue can be made by the cutting device 23. This step may be accomplished by the physician manipulating the first and second pull members 40, 42 so that the cutting deflector 28 moves back and forth to the positions shown in FIGS. 3B and 3C, thus causing the cutting device 23 to correspondingly move back and forth. The back and forth movement of the cutting device 23 may create a cutting motion that can be applied against the cancerous tissue to create an initial cut. The forceps 21 may then be advanced towards the mucosal flap created by the initial cut, or the forceps may be used first to grasp tissue that is to be cut with the cutting device 23. The forceps 21 can be manipulated open and closed by the similar method used to move the deflectors 26, 28 or other methods known in the art. Once the forceps 21 have closed around the mucosal flap and grasped it, the pull members 56, 58 may be manipulated by the physician to lift the mucosal flap away from the surrounding tissue. The physician may accomplish this step by pulling the third pull member 56 in a proximal direction, thus causing the forceps deflector 26 and the forceps 21 to move in the y-direction. The forceps 21 along with the mucosal flap may then be held in that position which provides the physician with a clear view of the cancerous tissue that needs to be excised. The cutting device 23 may then be manipulated in a back and forth cutting motion with the assistance of the pull members 40, 42 to create a series of small cuts in the tissue. Eventually, the forceps 21 may release the mucosal flap, and the endoscope 10 may be repositioned to a new location, where the process is repeated. The forceps 21 may be used to grab a new piece of the mucosal flap and the cutting device 23 may be used to create a new series of small cuts in the tissue. This process is repeated until the entire section of cancerous tissue has been excised from the surrounding healthy tissue. The endoscope 10, along with the rest of the device, may then be retracted from the patient's body. The removed cancerous tissue may also be removed from the patient's body with the use of the forceps 21 or other well-known retrieval tool.

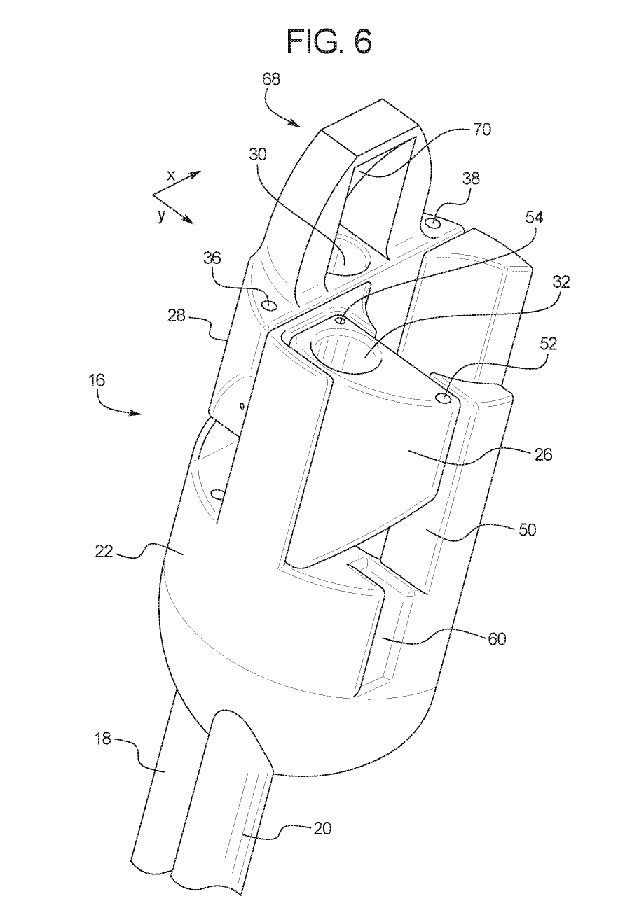

In the embodiment described above, the distal end of the deflectors 26, 28 are flush with, or end at point proximal to, the distal end of the housing 22. However, the designs of the deflectors 26,28 and housing 22 are not so limited. For example, FIG. 6 shows an embodiment where the cutting deflector 28 includes an extension 68 that extends distally past the distal end of the housing 22. The first working channel 30 may extend through the extension 68 such that the cutting device 23 exits the distal end of the first working channel 30 at a point further from the distal end of the endoscope 10, thereby potentially increasing visibility of the cutting deflector 28 when viewing it through the camera of the endoscope 10. In addition, the extension 68 may include a ramp 70 (shown in FIG. 6) where the distal end of the first working channel 30 terminates. The ramp 70 may alter the angle or direction at which the cutting device 23 exits the first working channel 30 by bending the distal end of the cutting device 23 with respect to the rest of the cutting deflector 28. For example, the ramp 70 may bend the distal end of the cutting device 45 degrees or more (or less) to allow easier access to additional areas of a body lumen. The ramp 70 may also increase the visibility of the cutting deflector 28 when viewing it through the camera of the endoscope 10, thereby allowing the operator to more easily use the device.

While the present disclosure is directed towards an ESD procedure, the above embodiments may be used in a variety of other medical procedures. Particularly, the above embodiments may be beneficial for use with scopes and treatment tools where greater flexibility and range of motion is desired. It should be understood also that the following claims are directed to the accessory device and do not require an endoscope to be present unless expressly and positively claimed.

Those of skill in the art will appreciate that embodiments not expressly illustrated herein may be practiced within the scope of the claims, including that features described herein for different embodiments may be combined with each other and/or with currently-known or future-developed technologies while remaining within the scope of the claims. Although specific terms are employed herein, they may be used in a generic and descriptive sense only and not for purposes of limitation unless specifically defined by context, usage, or other explicit designation. It is therefore intended that the foregoing detailed description be regarded as illustrative rather than limiting. It should be understood that the following claims, including all equivalents, are intended to define the spirit and scope of this invention. Furthermore, the advantages described above are not necessarily the only advantages of the invention, and it is not necessarily expected that all of the described advantages will be achieved with every embodiment. In the event of any inconsistent disclosure or definition from the present application conflicting with any document incorporated by reference, the disclosure or definition herein shall be deemed to prevail.

We claim:

1. A medical device, configured to facilitate a minimally invasive surgical procedure and comprising:
a housing comprising an attachment portion, the attachment portion configured to removably engage with a scope;
a first deflector engaged with the housing, the first deflector configured to engage with a distal end of a first treatment tool, the first deflector permitting movement of the distal end of the first treatment tool independent of the housing; and
a second deflector engaged with the housing, the second deflector configured to engage with a distal end of a second treatment tool, the second deflector permitting movement of the distal end of the second treatment tool independent of the housing.

2. The medical device of claim 1, further comprising:
a distal portion of a scope removably engaged with the attachment portion of the housing.

3. The medical device of claim 2, wherein:
the scope is friction force fit to the attachment portion of the housing.

4. The medical device of claim 1, wherein:
the housing, the first deflector, and the second deflector are configured such that actuating movement of the first deflector relative to the housing occurs along a first plane and actuating movement of the second deflector occurs along a second plane.

5. The medical device of claim 4, wherein:
the first plane is substantially orthogonal to the second plane.

6. The medical device of claim 1, further comprising:
a first accessory catheter attached to the housing;
a first working channel extending and providing a path of mechanical communication through the first accessory catheter, the housing, and the first deflector, wherein the first working channel is configured to removably receive and allow operation therethrough of a first treatment tool;
a second accessory catheter attached to the housing; and
a second working channel extending and providing a path of mechanical communication through the second accessory catheter, the housing, and the second deflector, wherein the second working channel is configured to removably receive and allow operation therethrough of a second treatment tool.

7. The medical device of claim 1, wherein:
the first deflector comprises a ramp in mechanical communication with a distal end of the first working channel, the ramp configured to bend the distal end of the first treatment tool with respect to the first deflector.

8. The medical device of claim 1, further comprising:
a first treatment tool comprising a cutting tool and a second treatment tool comprising a forceps, each disposed and operable through one of the first deflector and the second deflector.

9. The medical device of claim 1, wherein:
the first deflector comprises first and second pull members, the first pull member configured to rotate the first deflector in a first direction and the second pull member configured to rotate the first deflector in a second direction, wherein rotation of the first deflector is configured to cause corresponding rotation of the distal end of a first treatment tool, wherein the second direction is diametrically opposed to the first direction; and
the second deflector comprises third and fourth pull members, the third pull member configured to rotate the second deflector in a third direction and the fourth pull member configured to rotate the second deflector in a fourth direction, wherein rotation of the second deflector is configured to cause corresponding rotation of the distal end of a second treatment tool, wherein the third direction is diametrically opposed to the fourth direction.

10. The medical device of claim 9, further comprising:
a first treatment tool comprising a cutting device and a second treatment tool comprising a forceps, each disposed and operable through one of the first deflector and the second deflector.

11. The medical device of claim 9, further comprising:
a first accessory catheter attached to the housing;
a first working channel extending and providing a path of mechanical communication through the first accessory catheter, the housing, and the first deflector, wherein the first working channel is configured to removably receive and allow operation therethrough of the first treatment tool;
a second accessory catheter attached to the housing; and
a second working channel extending and providing a path of mechanical communication through the second accessory catheter, the housing, and the second deflector, wherein the second working channel is configured to removably receive and allow operation therethrough of the second treatment tool.

12. The medical device of claim 11, further comprising:
a first treatment tool comprising a cutting device and a second treatment tool comprising a forceps, each disposed and operable through one of the first deflector and the second deflector.

13. A method of removing tissue from a body lumen, comprising:
providing a scope cap, the scope cap comprising a housing comprising an attachment portion, the attachment portion configured to removably engage with a scope, a first deflector engaged with the housing, the first deflector configured to receive and allow operation therethrough of a distal end of a cutting device, the first deflector permitting movement of the distal end of the cutting device independent of the housing, and a second deflector engaged with the housing, the second deflector configured to receive and allow operation therethrough of a distal end of a forceps, the second deflector permitting movement of the distal end of the forceps independent of the housing;
advancing the scope cap into and through a body lumen until the scope cap is adjacent to a target tissue to be removed from a surrounding tissue;
inserting a cutting device through a first working channel that extends through and provides a path of mechanical communication through a first accessory catheter, the housing, and the first deflector, the first accessory catheter extending proximally from the housing;
inserting a forceps through a second working channel that extends through and provides a path of mechanical communication through a second accessory catheter, the housing, and the second deflector, the second accessory catheter extending proximally from the housing;
manipulating the second deflector into a position to grasp a portion of the target tissue with the forceps and separate the portion of the target tissue from the surrounding tissue;
manipulating the first deflector to create a series of incisions in the target tissue with the cutting device; and
repeating the steps of manipulating the second deflector and manipulating the first deflector with independent and/or concurrent operation of the cutting device and/or the forceps until the target tissue is excised from the surrounding tissue.

14. The method of claim 14, further comprising:
providing a scope.

15. The method of claim 15, further comprising:
attaching the scope cap to a distal end of the scope.

16. A system for performing a minimally invasive procedure, comprising:
a cap, the cap comprising:
a housing comprising an attachment portion, the attachment portion configured to removably engage with a scope;
a first deflector engaged with the housing, the first deflector configured to engage with a distal end of a first treatment tool, the first deflector permitting movement of the distal end of the first treatment tool independent of the housing; and
a second deflector engaged with the housing, the second deflector configured to engage with a distal end of a second treatment tool, the second deflector permitting movement of the distal end of the second treatment tool independent of the housing.

17. The system of claim 16, further comprising:
a scope, the cap removably attached to a distal end of the scope.

18. The system of claim 16, wherein:
the cap further comprises a first accessory catheter attached to the housing and a first working channel extending and providing a path of mechanical communication through the first accessory catheter, the housing, and the first deflector, wherein the first working channel is configured to removably receive and allow operation therethrough of the first treatment tool; and
the cap further comprises a second accessory catheter attached to the housing and a second working channel extending and providing a path of mechanical communication through the second accessory catheter, the housing, and the second deflector, wherein the second working channel is configured to removably receive and allow operation therethrough of the second treatment tool.

19. The medical device of claim 18, wherein:
the first deflector comprises a ramp in mechanical communication with a distal end of the first working channel, the ramp configured to bend the distal end of the first treatment tool with respect to the first deflector.

20. The system of claim 16, wherein:
the housing, the first deflector, and the second deflector are configured such that actuating movement of the first deflector relative to the housing occurs along a first plane and actuating movement of the second deflector occurs along a second plane.
